(19) United States
(12) Patent Application Publication
(10) Pub. No.: US 2016/0363995 A1
(43) Pub. Date: Dec. 15, 2016

(54) CIRCULAR LIGHT ELEMENT FOR ILLUMINATION OF CORNEA IN HEAD MOUNTED EYE-TRACKING

(71) Applicant: Seeing Machines Limited, Bradddon (AU)

(72) Inventor: Sebastian Rougeaux, Turner (AU)

(73) Assignee: Seeing Machines Limited

(21) Appl. No.: 15/177,290

(22) Filed: Jun. 8, 2016

(30) Foreign Application Priority Data
Jun. 12, 2015 (US) 2015902223

(51) Int. Cl. G06F 3/01; G06T 7/00; G02B 27/00; H04N 5/225; F21V 7/04; G02B 27/01

(52) U.S. Cl. CPC G06F 3/013; F21V 7/043; G02B 27/0172; G02B 27/0093; H04N 5/2256; G06T 7/0042; G02B 2027/0138; G02B 2027/014; G06T 2207/30201

(57) ABSTRACT

Embodiments of the present disclosure provide for apparatuses, methods, and systems for head-mounted eye gaze tracking system including: a camera for capturing images of a user's eyes; a light source for illuminating the user's eyes during the capturing of images; a light guide member for dispersing the light from the light source into a two-dimensional distribution for incidence onto the user's eyes; and a processor for processing the captured images to calculate the eye gaze direction of the user's eyes across a plurality of images by detecting a reflection of the two-dimensional distribution from the eyes.

This application claims priority under 35 U.S.C. §119 to Australian patent application 2015902223, filed Jun. 12, 2015, the contents of which are incorporated herein by reference.

### BRIEF DESCRIPTION OF THE FIGURES

Preferred embodiments of the disclosure will now be described, by way of example only, with reference to the accompanying drawings in which:

FIG. 1. illustrates a side view of an eye tracking system for a HMD including a light dispersing ring illumination source, a camera and image processor, according to an exemplary embodiment of the present disclosure;

FIG. 2 illustrates a plan view of the eye tracking system of FIG. 1;

FIG. 3 is a front view of the eye tracking system of FIG. 1 as seen by a user of the HMD, according to an exemplary embodiment of the present disclosure;

FIG. 4A illustrates a light ring and cross section, according to an exemplary embodiment of the present disclosure;

FIG. 4B shows a light dispersing ring shape and a light dispersing ring coupled to or in close proximity to an LED light source, according to an exemplary embodiment of the present disclosure;

FIG. 5 illustrates a camera view of a user's eye with the ring illumination source reflected from the users cornea, according to an exemplary embodiment of the present disclosure;

FIG. 6A illustrates a reflection on a user's cornea when the user is gazing straight ahead, according to an exemplary embodiment of the present disclosure;

FIG. 6B illustrates a reflection on a user's cornea when the user is gazing to the right, according to an exemplary embodiment of the present disclosure;

FIG. 6C illustrates a reflection on a user's cornea when the user is gazing to the left, according to an exemplary embodiment of the present disclosure;

FIG. 7 illustrates a flow chart diagram of one embodiment of the eye tracking system, according to an exemplary embodiment of the present disclosure; and

FIG. 8 is a schematic illustration of a pair of eyes imaged by a camera illustrating gaze rays and a point of regard, according to an exemplary embodiment of the present disclosure.

### DESCRIPTION OF EMBODIMENTS

The present disclosure relates to eye gaze tracking systems and in particular to a device for illuminating an eye in a head mounted eye gaze tracking system. Preferred embodiments of the disclosure are directed to eye gaze tracking systems in a Heads-up Display or other head mounted displays with eye tracking capabilities.

While some embodiments will be described herein with particular reference to the above applications, it will be appreciated that the disclosure is not limited to such a field of use, and is applicable in broader contexts.

Computing devices such as personal computers, laptop computers, tablet computers, cellular phones, and countless types of Internet-capable devices are increasingly prevalent in numerous aspects of modern life. Over time, the manner in which these devices are providing information to users is becoming more intelligent, more efficient, more intuitive, and less obtrusive.

The trend toward miniaturization of computing hardware, peripherals, as well as of sensors, detectors, and image and audio processors, among other technologies, has helped open up a field sometimes referred to as “wearable computing.” In the area of image and visual processing, it has become possible to consider wearable displays that place a very small image display element close enough to a wearer's (or user's) eye(s) such that the displayed image fills or nearly fills the user's or viewer's field of view, or appears as a normal sized image, such as might be displayed on a traditional image display device. The relevant technology may be referred to as “near-eye displays.”

Near-eye displays are fundamental components of wearable displays, also sometimes called “head-mounted displays” (HMDs). Use of the term “HMD” in this specification is intended to refer to any type of display device that is mounted to a user's head. These include, but are not limited to virtual or augmented reality headsets such as Oculus Rift™ or Magic Leap™, helmet mounted displays and eyewear displays such as Google Glass™. A HMD places a graphic display or displays close to one or both eyes of a wearer or user. To generate the images on a display, a computer processing system is typically used. Some HMDs, such as Samsung Gear VR™, pair with a smartphone to leverage the display and processor of that device to produce an augmented reality scene to a user. Displays of HMDs may occupy a wearer's entire field of view, or only occupy part of wearer's field of view.

Emerging and anticipated uses of wearable displays include applications in which users interact in real time with an augmented or virtual reality. Such applications can be mission-critical or safety-critical, as in a public safety or in an aviation setting. The applications can also be used in recreation, such as interactive gaming or entertainment.

While head-mounted display technologies have undergone significant developments, they have suffered from tradeoffs and limitations in capability. Among the tradeoffs and limitations, eye movement is often an overlooked aspect. Eye gaze tracking technology has proven to be useful in many different fields, including human-computer interfaces and assisting disabled people interact with a computer. Gaze tracking can also be used for industrial control, aviation, automotive, safety and many other applications.

Near-infrared light is often employed, as users cannot see this light and are therefore not distracted by it. Many eye tracking systems discard or remove glint or light directly reflected from a user's cornea to improve the reliability of eye pupil tracking. The light reflected from the eye has two major components. One component is light that has entered the eye and has been reflected back out from the retina. This light serves to illuminate the pupil of the eye from behind, causing the pupil to appear as a bright disk against a darker background. Another reflection component is a ‘glint’, which is a small, bright virtual image of the light source reflected from the front surface of the cornea. In many cases, the glint is an undesirable reflection component. An eye gaze tracking system determines the center of the pupil, and the change in the distance and direction between the two as the eye is rotated.

Commercially available eye gaze tracking systems are generally expensive and complicated. To be widely accepted and incorporated into mainstream commercial products, eye tracking technology should be relatively inexpensive, reliable, unobtrusive, and operator-friendly.

A primary objective of the preferred embodiments of the disclosure is to provide methods, systems, apparatus and devices for head mounted eye-tracking. In a particularly preferred embodiment, an eye tracking system is integrated into a head mounted display (HMD).

The eye tracking method and system incorporates a circular or round shaped infrared LED light source, camera or image capture device and processor or circuitry configured to analyze image data including image threshold, edge extraction and shape model curve fitting.

The disclosure may be used in a variety of applications including but not limited to gaming, user interface systems such as alternatives for mice or touch panels, medical systems including the collection of site direction information, military target recognition, and simulation of land, air, or water vehicles.

In accordance with a first aspect of the present disclosure there is provided a light dispersing member for an eye tracking device, the light dispersing member including a curved elongate tubular member having an internal surface that is at least partially covered in an at least partially reflecting material and a slot aperture extending at least partially longitudinally along the tubular member.

In one embodiment the curved elongate tubular member forms a torus. This embodiment preferably includes a light source disposed within the curved elongate tubular member for propagating light along the tubular member for output through the slot aperture.

In another embodiment the curved elongate tubular member forms a segment of a torus. The torus segment preferably subtends an angle of 270 degrees or greater.

The torus segment includes two ends and one end preferably includes an opening for receiving light from a light source. In another embodiment both ends include an opening for receiving light from one or more light sources.

In one embodiment the slot aperture is covered with a film that is at least partially transparent to infrared and/or visible light.

In one embodiment the curved elongate tubular member has a radius of curvature in the range of 15 mm to 25 mm.

In one embodiment the slot aperture has a width in the range 1 mm to 2 mm.

In one embodiment the curved elongate tubular member has a radial diameter in the range 1 mm to 10 mm.

In accordance with a second aspect of the present disclosure there is provided an eye tracking system including a light dispersing member according to any one of the preceding claims.

In accordance with a third aspect of the present disclosure there is provided a head-mounted eye gaze tracking system including:
a camera for capturing images of a user's eyes;
a light source for illuminating the user's eyes during the capturing of images;
a light dispersing member for dispersing the light from the light source into a two-dimensional distribution for incidence onto the user's eyes; and
a processor for processing the captured images to calculate the eye gaze direction of the user's eyes across a plurality of images by detecting a reflection of the two-dimensional distribution from the eyes.

In one embodiment the processor binarises the captured images by separating the pixels into one of two values based on a threshold pixel value.

In one embodiment the processor performs an edge extraction routine to extract the position of the two-dimensional distribution.

In one embodiment the processor performs a cornea pose fitting routine to the images based on a cornea shape model.

In one embodiment the processor calculates positions of the corneas of the user's eyes using the two-dimensional distribution.

In one embodiment the processor calculates positions of a center of each of the user's eyes and the eye gaze direction is extrapolated from the relative positions of the corneas and the centers of the eyes.

In one embodiment the light dispersing member is situated at a range of 10 mm to 30 mm from the user's corneas.

Preferably the two-dimensional distribution lies outside a field of view of the user.

In one embodiment the light source is disposed inside the light guide member. In another embodiment the light source is disposed adjacent the light guide member.

Preferably the light source includes one or more light emitting diodes.

Embodiments of the present disclosure relate to an eye tracking system integrated into a head mounted display (HMD) and provides eye-tracking without significant occlusion of a user's field of view. The described embodiments of the disclosure provide an improvement in the accuracy of eye tracking. Optionally, the eye tracking device may interface with a multitude of other systems or devices. Although the illumination source or light source in the description below is described as infrared or near-infrared light, the light source may be visible or invisible light at other wavelengths. The general preference for invisible light is to prevent user distraction or to reduce interference other unwanted visual light sources.

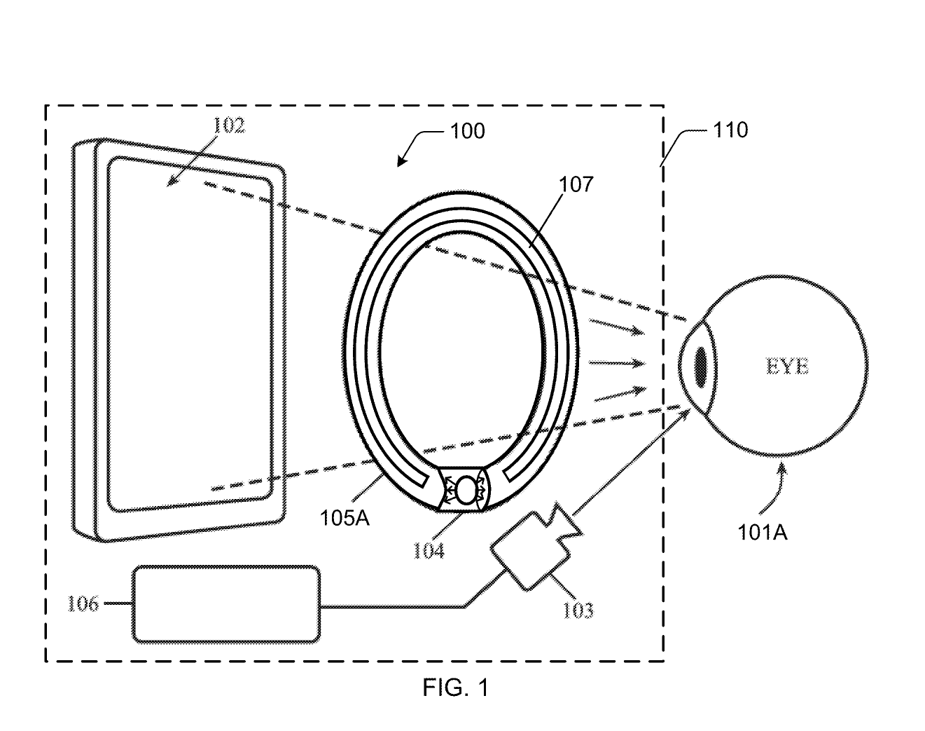

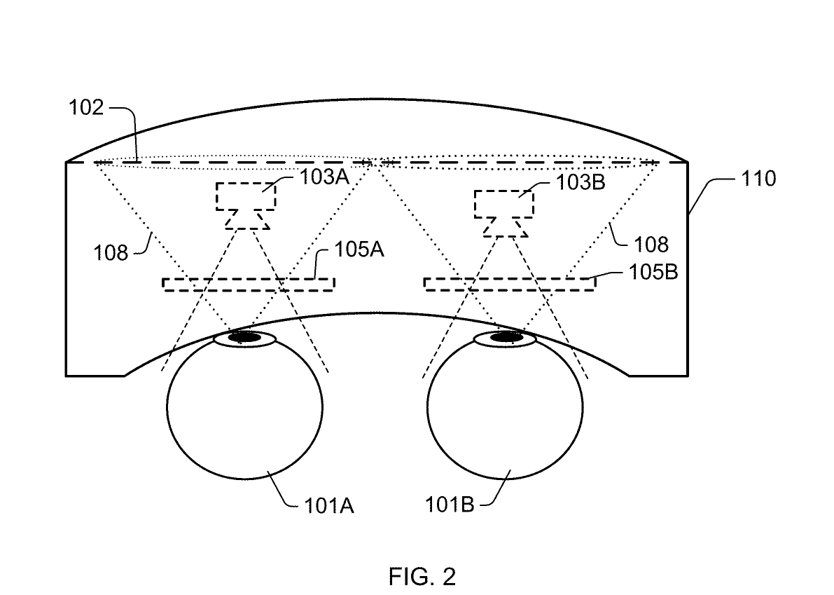

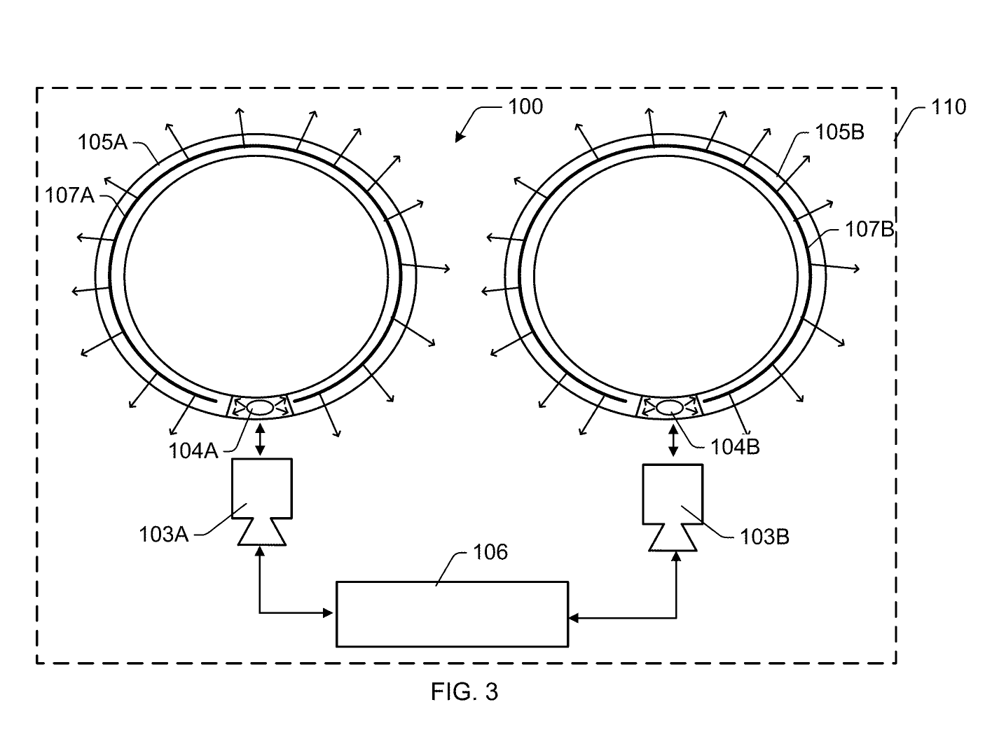

Referring initially to FIGS. 1 to 3, there is illustrated an exemplary embodiment of an eye tracking system 100 within a HMD 110. System 100 includes a pair of cameras 103A and 103B, illumination sources 104A and 104B and a vision processor 106. FIG. 1 illustrates a side view of system 100 showing a single eye while FIG. 2 illustrates a plan view of system 100 and FIG. 3 illustrates a front view of system 100 as viewed by a user of the HMD. In operation, a user's eye 101 views a scene, such as a display 102 while the cameras 103A and 103B capture images of the user's eyes 101A and 101B under illumination by illumination source 104. The illumination sources 104A and 104B includes one or more light sources disposed in or adjacent each of a pair of light dispersing members in the form of rings 105A and 105B, which will be described in detail below. The illumination sources 104A and 104B, which preferably includes one or more light emitting diodes (LEDs), is adapted to selectively emit electromagnetic radiation in the infrared or near-infrared wavelength range. The emitted radiation is directed by the light dispersing ring 107 onto the face and eyes of the user and reflections from the eyes are captured by cameras 103A and 103B. Cameras 103A and 103B includes a two dimensional array of detectors configured in a similar fashion to conventional digital cameras, wherein the detectors are sensitive to radiation in the infrared or near-infrared wavelength range so as to efficiently detect the reflected light. Radiation detected by cameras 103A and 103B is converted into electrical signals and sent to processing system 106 for conversion to digital images. Processing system 106 subsequently processes the images to perform eye gaze tracking of the user. In some embodiments, a single camera having a wide angle of view can be used in place of cameras 103A and 103B.

In one embodiment, vision processor 106 may be implemented with a microprocessor, or a plurality of microprocessors in conjunction with custom or specialized circuitry, executing program instructions or code stored in memory. The vision processor may also include memory, such as random access memory (RAM), read-only memory (ROM), electrically erasable programmable read-only memory (EEPROM), and other equivalent memory or storage systems as should be readily apparent to those skilled in the art.

Illumination sources 104 may be controlled by vision processor 106 or by another processor or controller, or within cameras 103A and 103B. Portions of the eye tracking system 100 may be integrated together or may be implemented in separate hardware and/or software or firmware. In each described embodiment, the cameras 103A and 103B or vision processor 106 may each, separately or together, generate control signals for any or each of other portions within the system 100. For example, synchronizing signals for cameras 103A and 103B are generated by vision processor 106 or, in another example, cameras 103A and 103B generate synchronizing signals for vision processor 106.

Light dispersing rings 105A and 105B are formed of a substantially torus shaped hollow tube comprised of a rigid material such as metal, glass or plastic. Light rings 105 are mounted within HMD 110 by an adhesive or suitable support frame depending on the design of HMD 110. In some embodiments, light rings 105 are mounted to lenses (not shown) of the HMD 110. As best illustrated in FIGS. 1 and 3, light rings 105 each include a slot opening 107 that extends at least partially along a curved longitudinal axis of the torus on a side facing the user's eyes 101A and 101B, as best illustrated in FIG. 1. An inner surface of the torus shaped tube is formed of an infrared reflective material such as aluminium or mirrored glass to diffusely or specularly reflect light from illumination sources 104A and 104B along the tube. The internally reflected light is output through slot opening 107 at various longitudinal positions and is incident on the eyes 101A and 101B to form a substantially circular image on the eyes. In some embodiments, slot opening 107 is covered in a material transparent to the emitted infrared or near-infrared radiation, such as glass, to seal the interior of rings 105 from external elements such as dust.

As illustrated in FIGS. 1 and 2, light dispersing rings 105 are positioned proximate to or near the user's eye. However, rings 105 are positioned at a suitable distance so that the projected light is outside of the user's field of view 108 allowing the user to see an unobstructed view of the scene, image, or display 102. In one embodiment, rings 105 are disposed at positions in the approximate range of 1 cm to 3 cm away from the user's cornea. Cameras 103A and 103B are preferably mounted proximate to the light rings 105 at a position below and between rings 105, as illustrated in FIGS. 1 and 3. In other embodiments, cameras 103A and 103B is disposed at other locations such as above or to one side of rings 105. In some embodiments, the user's eyes 101 are imaged by more than one camera.

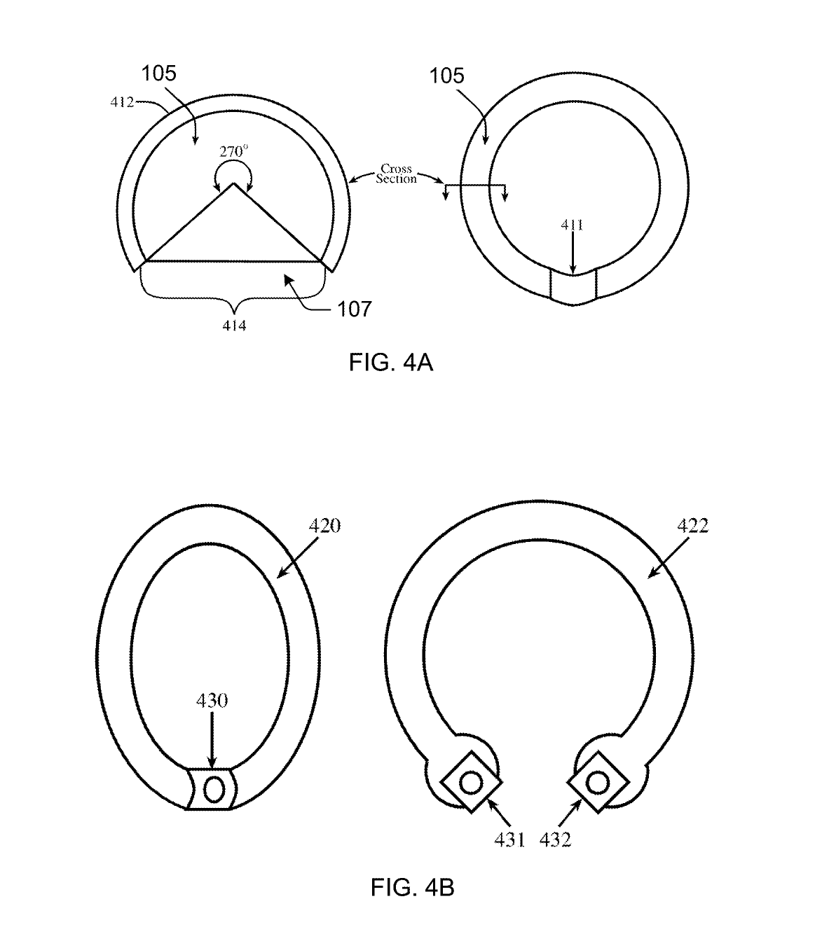

Referring now to FIGS. 4A and 4B, in one exemplary embodiment of light dispersing ring 105, a single infrared or near infrared LED 430 is optically coupled to ring 105, which is configured to allow infrared or near infrared light to pass through its plastic or other compositional material. The ring 105 includes an internally reflective coating 412 and an indentation or physical LED interface 411 in which LED 430 is housed. The structure of reflective coating 412 is adapted to project a generally or approximately uniform light ring in the direction toward a user's eye or cornea. In one embodiment, the ring diameter is 38 mm. However, in other embodiments the ring diameter is in the range of 30 mm to 45 mm, inclusive. In preferred embodiments, the ring has a circular or near circular shape. However, in another embodiment, the light dispersing ring is elliptical, as illustrated by ring 420 in the left panel of FIG. 4B. In additional light ring embodiments, the shape for example may be a rounded rectangle, rounded square, rounded diamond, rounded triangle, or other similar two dimensional shapes.

Referring to FIG. 4A, in a cross sectional view of the light dispersive ring 105, the reflective coating 412 surrounds a predetermined portion of ring 105. In FIG. 4A, an approximate reflective coating coverage of 270° is shown although a range of approximately 135° to slightly under 360° may be implemented. The cross sectional view in FIG. 4A shows a round cross section with a flat light emission area through opening 414 of the longitudinal emission slot 107. A variety of different shapes may be used for the ring and light emission area. The ring 105 compositional material allows infrared or near infrared light to pass through the ring material and the light is reflected and scattered by the reflective coating throughout the ring interior until the light exits through opening 414, providing a portion of an approximate circle or an approximate circular illumination source from the single LED. Using a light ring proximate to a user's cornea or eye reduces the required light brightness to detect a corneal reflection, resulting in reduced cost of the system and increased efficiency.

In alternative embodiments, such as in ring 422 of FIG. 4B, a plurality of infrared or near infrared LEDs 431, 432 are used to improve either illumination levels or illumination uniformity. The plurality of LEDs may be optically coupled to the ring 422 at a single position on the light ring or the plurality of LEDs may be optically coupled to the ring 422 in a plurality of positions located throughout the ring 422. Additionally, in other embodiments, multiple light rings may be configured concentrically or in other patterns with the objective of increasing accuracy, performance or other improvements to an eye tracking system.

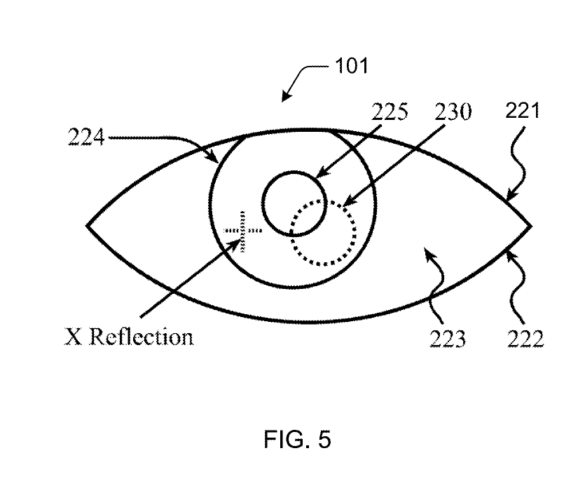

In operation, light dispersive rings 105 project a ring of light from slot 107 toward a user's eye, and cameras 103A and 103B and vision processor 106 collectively monitor the reflected position of the light ring in images from the cornea surface. Referring now to FIG. 5, an exemplary camera view or camera perspective of the user's eye is shown. Edges of the upper and lower eyelids 221, 222, sclera 223, iris/cornea 224 and pupil 225 are illustrated with a resulting light ring reflection 230 from the light ring 105 on the user's eye. The light ring reflection will change position within the camera image if the user's eye changes position indicating the direction of the user's gaze.

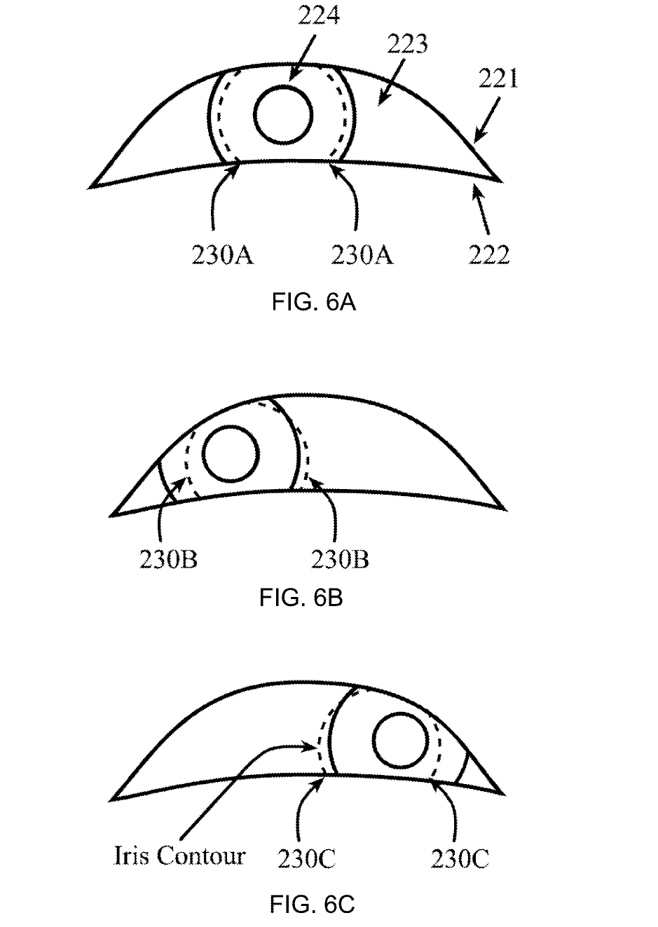

Referring to FIGS. 6A, B, C, exemplary camera views or camera perspectives of a user's eye are shown. In FIG. 6A, an example of a user looking straightforward has a light ring reflection 230A generally located proximate to the center of the eye opening. As a user looks to the right and left, as respectively illustrated in FIG. 5B and FIG. 5C, a light ring reflection 230B, 230C generally follows a user's gaze direction. The ring reflection is therefore used as a reference point in relation to the moving pupil for accurate eye or pupil tracking. Optionally, a reference model may be developed to generate an adjusted light ring reflection position to improve accuracy.

In FIGS. 5 and 6A-C, a light ring reflection 230 illustrated is a full approximate circle. However, the upper and lower eyelids 221, 222 are not likely to be reflective and therefore there may be an occlusion or a partial ring reflection as illustrated in FIGS. 6A-C. With a partial light ring reflection, a single arc or line segment or a plurality of arcs or line segments are identified and the center of the reflected light ring circle is calculated and identified. Furthermore, the corneal reflection of the light ring circle will generally be distorted into an elliptical shape in the horizontal and/or vertical direction from the curved geometry of the eye. The elliptical or distorted shape of the reflection may be adjusted by camera or image processor routine, operation, analysis, or algorithm. The amount of distortion imparted by the eye provides additional input for calculating the eye gaze depending on the eye model used. The eye is preferably illuminated with at least 30% of a ring profile to provide a sufficient reflection profile for eye tracking.

Advantages in having a ring of light (or other two dimensional distribution) as an illumination source includes providing a wider and larger area of reflection from the user's cornea and the ability to track the user's gaze or eye position without pupil tracking. Tracking by reflection provides a simple geometric eye model and improved processor performance by reducing a computational load in comparison to pupil tracking.

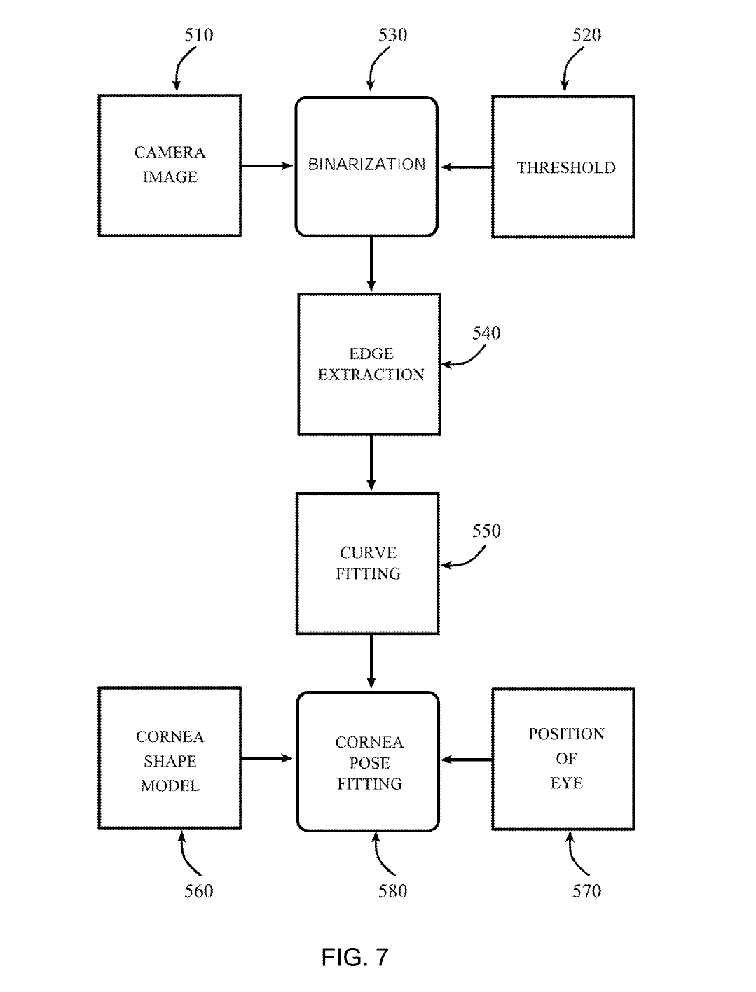

Referring now to FIG. 7, there is illustrated an exemplary method 500 of estimating eye gaze direction of user's eye using system 100. At step 510 a camera image containing a view of the user's eye and light ring reflections are generated by the camera. A pixel threshold routine, operation, analysis, or algorithm is performed at steps 520 and 530, either directly by the cameras 103A and 103B or the image processing device 106. Preferably a threshold pixel value is used and, in one threshold embodiment, a fixed predetermined pixel threshold value is used and in another threshold embodiment, a dynamic pixel threshold value is determined by the cameras 103A and 103B or the image processing device 106. For example, a dynamic threshold value may be determined from an average or an adjusted weighted value that is derived from at least one camera image.

A binary image from the camera image is generated at step 530 either by the cameras 103A and 103B or the image processing device 106. Generally, the binary image shows only the ring reflection or enhances the ring reflection from the camera image. Generating a binary image as an approximate 100% black and 100% white representation reduces the pixel representation digits from multiple digital bits or words to a single bit or reduced pixel depth, therefore reducing the amount of memory or memory size required to store an image. This device, method or process provides a simple and efficient model to track a user's gaze or eye position.

After a binary image is generated, an edge extraction routine, operation, analysis, or algorithm is performed at step 540 using the binary image to identify edges in the image. As the binarized ring reflection represents a sharp edge in the binary image, the ring reflection position can be identified in the image. A curve fitting routine, operation, analysis, or algorithm is then performed at step 550 on the identified ring reflection shape to determine the curvature and center of the ring. This serves to define a reference point based on the extracted ring reflection image within the camera image. Optionally, the reference point is compared to a correlation model providing an adjusted reference point. Steps 540 and 550 are implemented as an ‘optimization’ procedure. The parameters (state) of the optimization procedure are the 3D location of the cornea. The errors (residuals) of the optimization are the distance between the estimated reflection and the measured reflection by the camera. The end result of the optimization procedure is the best 3D cornea center location that matches the measured reflection in the image.

Next, the detected 3D cornea center position is fit to the eye geometry to determine a gaze direction vector. A corneal shape model is applied at step 560 and a ring reflection reference point or adjusted reference point is applied at step 570. The reference point indicates the position or pose of the user's eye and is used by a cornea pose fitting routine, operation, analysis, or algorithm performed at step 580.

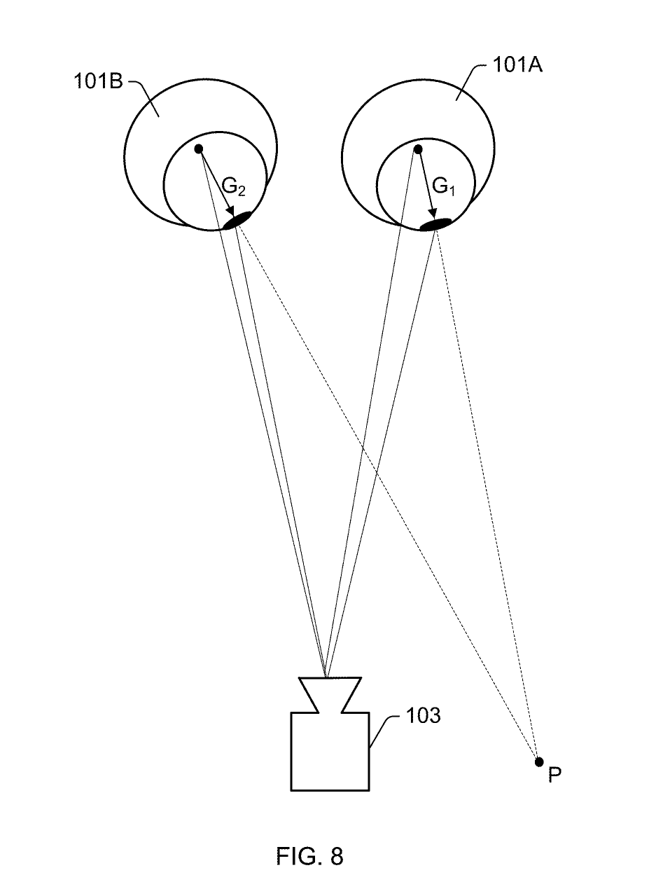

The eye-gaze estimation technique relies on the measurement of the eyeball center of rotation, and the detected 3D cornea center. The eye-gaze direction for each eye is computed as the ray passing through these two points, as illustrated as vectors G₁ and G₂ in FIG. 8. The eyeball center position is determined by a calibration procedure described as follows.

Initially the optimal eyeball center location is calculated using several frames of captured images where the user gazes at different locations within the camera's field of view. When a user is looking directly into the camera lens, the ray from the camera center to the center of the cornea passes through the eyeball center of rotation. The user then looks straight towards the camera for a short period (several frames of captured images) while moving their head around inside the camera's field of view. The system records measurements of 3D cornea center position during this period. Using these measurements of 3D cornea center position, the eye tracking system is able to estimate the center of eyeball rotation by calculating the least-squares intersection of a number of rays. In this estimation, the camera location is assumed to be fixed with respect to the user's head so that the center of rotation of the eyeball is also assumed to be fixed with respect to the camera.

The calculation is performed for both eyes, and may be improved by using the technique with more than one camera.

The eyeball center and 3D cornea center position are fed to the cornea pose fitting routine at step 580 which applies geometrical constraints based on a cornea shape model to derive eye gaze direction vectors. In an exemplary corneal shape model, an eye is modeled as a sphere of about 24 mm diameter having a spherical corneal radius of about 15 mm diameter. Eyes of different adult users will have variations from these typical values, but, generally, individual dimensional values do not vary significantly, and a standard or adjusted corneal shape map may be used as a general model. Note that spherical or non-spherical cornea models, including parabolic models, are known in the art and may be optionally employed by the present disclosure. The position of the user eye or cornea will therefore also provide an accurate position of the user iris and pupil in tracking or eye position or gaze direction.

The output of the eye gaze tracking estimation method 500 is the eye gaze vectors G₁ and G₂ and optionally a point of regard P, as illustrated in FIG. 8. The method may be performed continuously, semi-continuously or routinely to perform tracking of the user's eye gaze over time.

The disclosure described above provides for a head-mounted display having an integrated eye-tracking system that does not obstruct a user's view. In addition, an illumination or light source in an approximate ring shape, using a corneal reflection or glint, increases eye tracking accuracy, is less susceptible to occlusion and refractions and provides an efficient and cost effective corneal or user gaze model. A user's gaze or eye position is tracked without the requirement for tracking the user's pupil.

The present disclosure is applicable to any type of head mounted tracking system including Head Mounted Displays (HMD), glasses (e.g. Google Glass™) or helmets (such as for pilots). The light dispersing ring may be retrofitted to pre-manufactured systems or manufactured integrally within new systems.

In addition, and optionally, calibration routines, operations, analyses, or algorithms to refine the shape model may be implemented within the eye tracking system. The use of a light dispersing ring also requires less calibration points to improve and develop a very accurate eye tracking system. Another advantage of using a light ring is corneal refraction distortion is reduced or eliminated.

It should be appreciated that in the above description of exemplary embodiments of the disclosure, various features of the disclosure are sometimes grouped together in a single embodiment, FIG., or description thereof for the purpose of streamlining the disclosure and aiding in the understanding of one or more of the various inventive aspects. This method of disclosure, however, is not to be interpreted as reflecting an intention that the claimed disclosure requires more features than are expressly recited in each claim. Rather, as the following claims reflect, inventive aspects lie in less than all features of a single foregoing disclosed embodiment. Thus, the claims following the Description are hereby expressly incorporated into this Description, with each claim standing on its own as a separate embodiment of this disclosure.

Furthermore, while some embodiments described herein include some but not other features included in other embodiments, combinations of features of different embodiments are meant to be within the scope of the disclosure, and form different embodiments, as would be understood by those skilled in the art. For example, in the following claims, any of the claimed embodiments can be used in any combination.

In the description provided herein, numerous specific details are set forth. However, it is understood that embodiments of the disclosure may be practiced without these specific details. In other instances, well-known methods, structures and techniques have not been shown in detail in order not to obscure an understanding of this description.

Thus, while there has been described what are believed to be the preferred embodiments of the disclosure, those skilled in the art will recognize that other and further modifications may be made thereto without departing from the spirit of the disclosure, and it is intended to claim all such changes and modifications as falling within the scope of the disclosure. For example, any formulas given above are merely representative of procedures that may be used. Functionality may be added or deleted from the block diagrams and operations may be interchanged among functional blocks. Steps may be added or deleted to methods described within the scope of the present disclosure.

What is claimed is:

1. A light dispersing member for an eye tracking device, the light dispersing member including a curved elongate tubular member having an internal surface that is at least partially covered in an at least partially reflecting material and a slot aperture extending at least partially longitudinally along the tubular member.

2. A light dispersing member according to claim 1 wherein the curved elongate tubular member forms a torus.

3. A light dispersing member according to claim 2, further comprising a light source disposed within the curved elongate tubular member for propagating light along the tubular member for output through the slot aperture.

4. A light dispersing member according to claim 1 wherein the curved elongate tubular member forms a segment of a torus.

5. A light dispersing member according to claim 4 wherein the torus segment subtends an angle of 270 degrees or greater.

6. A light dispersing member according to claim 4 wherein the torus segment includes two ends and one end includes an opening for receiving light from a light source.

7. A light dispersing member according to claim 6 wherein both ends include an opening for receiving light from one or more light sources.

8. A light dispersing member according to claim 1 wherein the slot aperture is covered with a material that is at least partially transparent to at least one of infrared and visible light.

9. A light dispersing member according to claim 1 wherein the curved elongate tubular member has a radius of curvature in the range of 15 mm to 25 mm.

10. A light dispersing member according to claim 1 wherein the slot aperture has a width in the range 1 mm to 2 mm.

11. A light dispersing member according to claim 1 wherein the curved elongate tubular member has a radial diameter in the range 1 mm to 10 mm.

12. An eye tracking system including a light dispersing member including a curved elongate tubular member having an internal surface that is at least partially covered in an at least partially reflecting material and a slot aperture extending at least partially longitudinally along the tubular member.

13. A head-mounted eye gaze tracking system including:
a camera for capturing images of a user's eyes;
a light source for illuminating the user's eyes during capturing of images;
a light dispersing member for dispersing light from the light source into a two-dimensional distribution for incidence onto the user's eyes; and
a processor for processing captured images to calculate eye gaze direction of the user's eyes across a plurality of images by detecting a reflection of the two-dimensional distribution from the eyes.

14. A system according to claim 13 wherein the processor binarises the captured images by separating the pixels into one of two values based on a threshold pixel value.

15. A system according to claim 14 wherein the processor performs an edge extraction routine to extract the position of the two-dimensional distribution.

16. A system according to claim 15 wherein the processor performs a cornea pose fitting routine to the images based on a cornea shape model.

17. A system according to claim 16 wherein the processor calculates positions of the corneas of the user's eyes using the two-dimensional distribution.

18. A system according to claim 17 wherein the processor calculates positions of a center of each of the user's eyes and the eye gaze direction is extrapolated from the relative positions of the corneas and the centers of the eyes.

19. A system according to claim 13 wherein the light dispersing member is situated at a range of 10 mm to 30 mm from the user's corneas.

20. A system according to claim 13 wherein the two-dimensional distribution lies outside a field of view of the user.
